(19) United States
(12) Patent Application Publication
(10) Pub. No.: US 2021/0315818 A1
(43) Pub. Date: Oct. 14, 2021

(54) CANNABINOID BASED EMULSION SYSTEMS FOR INFUSED NON-AQUEOUS COMPOSITIONS

(71) Applicant: HEXO OPERATIONS INC., Ottawa (CA)

(72) Inventors: Max Alsayar, Ottawa (CA); Justin Conway, Carignan (CA); Francois Chouinard, Blainville (CA); Denis Keseris, Baie d'Urfe (CA); George Elvira, Montreal (CA); Stephan Georgiev, Saint-Hubert (CA); Walter Chan, Ottawa (CA)

(21) Appl. No.: 17/269,975

(22) PCT Filed: Aug. 20, 2019
(86) PCT No.: PCT/CA2019/051139

§ 371 Date: Feb. 19, 2021

Related U.S. Application Data
(60) Provisional application No. 62/719,926, filed on Aug. 20, 2018.
(60) Provisional application No. 62/722,422, filed on Aug. 24, 2018.
(60) Provisional application No. 62/725,142, filed on Aug. 30, 2018.

(51) Int. Cl. A61K 9/107; A61K 31/352; A61K 47/14; A61K 47/22; A61K 47/18; A61K 47/24; A61K 47/26; A61K 9/00; A23D 7/005; A23D 7/04; A23L 33/115; A23L 33/105; A23G 3/48; A23G 3/40; A23K 10/30; A23K 20/158; A23L 2/52

(52) U.S. Cl. CPC A61K 9/1075; A23V 2002/00; A61K 47/14; A61K 47/22; A61K 47/183; A61K 47/24; A61K 47/26; A61K 9/0056; A23D 7/0053; A23D 7/04; A23L 33/115; A23L 33/105; A23G 3/48; A23G 3/40; A23K 10/30; A23K 20/158; A23L 2/52; A61K 31/352

(57) ABSTRACT

The present disclosure relates to a cannabinoid based emulsification system for infusing a non-aqueous composition with a cannabinoid, the emulsification system comprising: a) at least one cannabinoid in a carrier oil; b) a plurality of emulsifiers having a targeted combined HLB value; and c) a targeted plurality of emulsifiers to oil ratio; wherein b) and c) so that at least 10 wt % of the cannabinoid is solubilized in the oral cavity within 30 minutes after a subject consumes the non-aqueous composition, based on the total weight of the cannabinoid in the non-aqueous composition. Methods of making and using such emulsification systems, and cannabinoid concentrate compositions and cannabis infused non-aqueous compositions comprising such emulsification systems to form beverages, human and pet edibles and confectionaries are also encompassed by the present disclosure.

### TECHNICAL FIELD

The present disclosure relates to cannabinoid based self-emulsification systems which are suitable for infusion of the cannabinoid into non-aqueous compositions useful to form a large variety of cannabis infused products (e.g., human or pet edibles, confectionaries). Method of making and using such emulsification systems, and cannabinoid concentrate compositions and cannabis infused non-aqueous compositions comprising such self-emulsification systems are also encompassed by the present disclosure.

### BACKGROUND

Cannabis infused human or pet edibles, and/or confectionaries are expected to grow in popularity due to the existing and/or expected legalization of these product forms in Canada and other countries (e.g., United States) globally. As a result, attention has turned to how to prepare industrial scale quantities of these products to meet consumer demands. One approach is to provide a concentrated pre-mix formulation of the cannabis extract that could be easily shipped to a manufacturer. The manufacturer would then dilute the concentrated pre-mix formulation into different product bases to form a large variety of different human edibles (e.g., chewing or bubble gums, mints) or pet edibles (e.g., pet food, pet chew) and/or confectionaries (e.g., lozenges) ready for commercial sale and consumption.

A key challenge here is to ensure that the cannabinoids are sufficiently solubilized in the concentrated pre-mix formulation all the way to the final cannabis infused products. This may be difficult because cannabis formulations are typically highly lipophilic (i.e., fat-loving) and have poor aqueous solubility (i.e., essentially water insoluble). Another challenge is the requirement that the solubilized cannabinoids is stable through-out the entire large scale production process and up to the point of consumption. The solubility of insoluble cannabis has been shown to be improved with the aid of emulsifiers to enable a dispersion of particles within a non-miscible solution. The use of emulsifiers also allows for the cannabinoids to appear evenly distributed throughout the formulations. Thus, ideally the concentrated pre-mix formulation would take the form of a composition comprising a cannabinoid solubilized in a carrier oil and then microencapsulated in an emulsion. This however has proven to be quite a challenging endeavour in view of the following considerations.

Firstly, to meet the expected user experience demands from consumers, the cannabis infused products should be able to satisfy some or preferably all of the following requirements: (i) improved water solubility of the cannabinoids to maximize the consumable limits of the cannabis (e.g., 10 mg of cannabis per beverage package for Canada), (ii) storage stability over the normal expected shelf-life (e.g., at least 6 months), (iii) transport stability over varying travel conditions (e.g., extreme temperatures, excessive agitation, etc.), (iv) clear physical appearance (for clear products) or no discoloration (for opaque products) and/or no adverse visual effects (e.g., ringing, creaming, etc.), (v) pleasant organoleptic properties (e.g., pleasing taste and smell), and (vi) slow and/or delayed release of the cannabinoids upon consumption by a subject.

Secondly, previous attempts to solubilize hydrophobic cannabis extracts into non-aqueous compositions have yielded consumer negative results due to incompatibilities between various properties of the emulsification system and components of the product per se. As a result, it can be quite difficult to formulate a cannabis extract present in a carrier oil into a solid or dried product form. For example, with a solid edible product like a chewing or bubble coat, particularly with a hard out coating, it is difficult to incorporate the cannabinoid in the carrier oil even when it has been spray dried.

While attempts have been made in the art to solubilize hydrophobic cannabis extracts into non-aqueous compositions using various emulsification approaches, there have been reports of inconsistent and/or poor results in terms of solubility, stability (transport and/or storage), and/or organoleptic properties obtained from such prior emulsification approaches. Furthermore, these previous emulsification approaches fail to provide a consistent consumer experience over a large variety of products due to their different components. This is a problem because it would force the manufacturer to formulate multiple cannabinoid based emulsification systems when infusing with a variety of products, which is undesirable as it would tend to increase costs, and decrease ease of manufacture.

Thus, there still remains a need for an improved cannabinoid based emulsification system having some or all of the desired properties as discussed above for large scale production of cannabis infused non-aqueous consumable good products. In particular, it is desirable that the cannabinoid based emulsification system provides good water solubility of the cannabinoids, for example, in the mouth of a subject upon consumption of the non-aqueous composition comprising the emulsification system. It is also desirable that the cannabinoid based emulsification system of the present disclosures provides superior performance in terms of odor, taste profile, and/or appearance in the consumable good products.

### SUMMARY

This Summary is provided to introduce a selection of concepts in a simplified form that are further described below in the Detailed Description. This Summary is not intended to identify key aspects or essential aspects of the claimed subject matter.

The inventors have developed cannabinoid based emulsification systems that are surprisingly capable of overcoming at least some or preferably all of the disadvantages of solubility, stability, clarity and organoleptic properties associated with the consumer experience, as described herein above. In particular, the emulsification systems of the present disclosure allow for good water solubility of the hydrophobic cannabis extracts once a subject consumes the non-aqueous composition comprising said emulsification systems. The emulsification systems also allow for enhanced stability, preferably over the normal shelf-life of the non-aqueous composition. Advantageously, the cannabinoid based self-emulsification systems of the present disclosure may have broader compatibility with a variety of product bases and therefore may provide a more consistent consumer experience over a larger variety of product bases.

As embodied and broadly described herein, the present disclosure relates to a cannabinoid based emulsification system for infusing a non-aqueous composition with a cannabinoid, the emulsification system comprising: a) at least one cannabinoid in a carrier oil; b) a plurality of emulsifiers having a targeted combined Hydrophile-Lipophile Balance (HLB) value; and c) a targeted plurality of emulsifiers to oil ratio; wherein b) and c) operate so that at least 10 wt %, preferably at least 20 wt %, preferably at least 30 wt %, or preferably at least 50 wt % of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

As embodied and broadly described herein, the present disclosure relates to a cannabinoid based emulsification system for infusing a non-aqueous composition with a cannabinoid, the emulsification system comprising: a) at least one cannabinoid in a carrier oil; b) a plurality of emulsifiers having a targeted combined Hydrophile-Lipophile Balance (HLB) value; and c) a targeted plurality of emulsifiers to oil ratio; wherein b) and c) operate so that at least 1 mg, preferably at least 2 mg, or preferably at least 5 mg of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

As embodied and broadly described herein, the present disclosure also relates to a process for manufacturing a cannabinoid based emulsification system as described herein, comprising: a) selecting at least one cannabinoid in the carrier oil; b) selecting a plurality of emulsifiers; and c) mixing the emulsifiers with the cannabinoid.

As embodied and broadly described herein, the present disclosure also relates to a cannabinoid concentrate composition in the form of an emulsion, the composition comprising a cannabinoid based emulsification system as described herein, and the emulsion having a particle size distribution (PSD) of less than 1000 nm.

As embodied and broadly described herein, the present disclosure also relates to a non-aqueous composition product comprising the cannabinoid concentrate composition as described herein.

As embodied and broadly described herein, the present disclosure also relates to a process to make the cannabinoid concentrate composition as described herein comprising the steps of: a) providing an oil phase comprising the cannabinoid extract; b) preparing an aqueous phase comprising water and optionally other ingredients; c) incorporating the emulsification system as described herein either in the oil phase or the aqueous phase; and d) dispersing the oil phase in the aqueous phase to form an emulsion, preferably a nano-emulsion.

All features of exemplary embodiments which are described in this disclosure and are not mutually exclusive can be combined with one another. Elements of one embodiment can be utilized in the other embodiments without further mention. Other aspects and features of the present disclosure will become apparent to those ordinarily skilled in the art upon review of the following description of specific embodiments in conjunction with the accompanying FIGURE.

### BRIEF DESCRIPTION OF THE DRAWINGS

While the specification concludes with claims particularly pointing out and distinctly claiming the invention, it is believed that the invention will be better understood from the following description of the accompanying FIGURE wherein like numerals are employed to designate like parts throughout the same.

FIG. 1 shows a flow diagram illustrating a process for producing a cannabinoid based emulsification system in accordance with a non-limiting embodiment of the present disclosure.

### DETAILED DESCRIPTION

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

As used herein, terms of degree such as “about”, “approximately” and “substantially” mean a reasonable amount of deviation of the modified term such that the end result is not significantly changed. These terms may refer to a measurable value such as an amount, a temporal duration, and the like, and are meant to encompass variations of +/−0.1% of the given value, preferably +/−0.5%, preferably +/−1%, preferably +/−2%, preferably +/−5% or preferably +/−10%.

As used herein, articles such as “a” and “an”, are understood to mean one or more of what is claimed or described.

As used herein, the term “non-aqueous composition” as used herein means a composition in which no water is intentionally added as a component or ingredient of the composition. In some embodiments, the composition lacks an aqueous environment in said composition. In some embodiments, the composition is one which contains no more than 1% by weight of water. In some embodiments, the non-aqueous composition contains less than 0.5%, less than 0.25%, less than 0.1%, less than 0.05% or less than 0.01% by weight water. It is understood that “less than” a certain percentage of water refers to from zero to the specified amount, within acceptable ranges of the detection of water by instrumentation known to those skilled in the art.

As used herein, the terms “comprises”, “comprising”, “include”, “includes”, “including”, “contain”, “contains” and “containing” are meant to be non-limiting, i.e., other steps and other sections which do not affect the end of result can be added. The above terms encompass the terms “consisting of” and “consisting essentially of”.

As used herein, the expression “delayed release” in the context of the cannabinoid means release of a cannabinoid in a manner such that release of the cannabinoid in vivo (i.e., oral cavity) is delayed in comparison to an immediate release from an immediate release compartment.

All non-ionic emulsifiers consist of a molecule that combines both hydrophilic and lipophilic groups. As used herein, the term “Hydrophile-Lipophile Balance (HLB) value” refers to the balance of the size and strength of the hydrophilic (water-loving or polar) and the lipophilic (oil-loving or non-polar) groups of the non-ionic emulsifier. However, as used herein, HLB value is not limited to its application to non-ionic emulsifiers alone. While HLB value has a specific meaning for non-ionic emulsifiers, its meaning can be extrapolated to other emulsifiers, regardless of whether it is ionic or non-ionic, as a general indicator of the hydrophilicity and lipophilicity. Therefore, it is contemplated that other types of emulsifiers may be useful within the scope of the present disclosure. For example, suitable emulsifiers may also be selected from the group consisting of anionic, cationic and amphoteric emulsifiers.

The HLB value is also an indication of the solubility of the emulsifiers. For example, conventional understanding is that a emulsifier having a high HLB value (i.e., 8-18 according to HLB:ICI Americas Inc., “The HLB System a Time Saving Guide to Emulsifier Selection”, Chemmunique, March 1980) will tend to be water-soluble and is used when it is desired that the final product exhibit aqueous characteristics, i.e., to dilute readily with water.

The HLB value for a given emulsifier is generally known by those skilled in the art or may be calculated using the Griffin's Mathematical Method (HLB=20×((Mₕ/M), wherein Mₕ=molecular weight of hydrophilic groups; M=molecular weight of the whole molecule). Alternatively, HLB values for a particular emulsifier may be determined by dividing the hydrophilic molecular weight percentage of the compound by 5.

Alternatively, the HLB values for emulsifiers may be listed in Kirk-Othmer, Encyclopedia of Chemical Technology, third edition 1979, vol. 8, page 913; and HLB:ICI Americas Inc., “The HLB System a Time Saving Guide to Emulsifier Selection”, Chemmunique, March 1980.

As used herein, the term “targeted combined HLB value” refers to the HLB values which correspond not to a single emulsifier but the resulting HLB value of the blend of two or more emulsifiers in the emulsification system needed to achieve a certain desired outcome. As discovered by the inventors, the targeted combined HLB value of a blend of emulsifiers is an excellent indication of what the emulsification system will do, that is, whether it will make emulsion, the type of emulsion (e.g., oil-in-water) and the ability to solubilize the cannabinoids. For example, it is desirous to select a targeted combined HLB value for the emulsification system so that when it is formulated into a non-aqueous composition it operates in the solubilization of at least 10 wt %, preferably at least 20 wt %, preferably at least 30 wt %, preferably at least 50 wt %, or preferably at least 100 wt % of the cannabinoid in the oral cavity within 30 minutes, preferably within 20 minutes, or preferably within 10 minutes after a subject consumes the non-aqueous composition, based on the total weight of the cannabinoid in the non-aqueous composition. Alternatively, the targeted combined HLB value for the emulsification system can be selected so that when it is formulated into a non-aqueous composition it operates in the solubilization of at least 1 mg, preferably at least 2 mg, preferably at least 5 mg, or preferably at least 10 mg of the cannabinoid in the oral cavity within 30 minutes, preferably within 20 minutes, or preferably within 10 minutes after a subject consumes the non-aqueous composition.

As used herein, the term “targeted plurality of emulsifiers to oil ratio” refers to a measure of the level of emulsifiers to oil in the emulsification system that a formulator wishes to maintain for the emulsification system so that when it is formulated into a non-aqueous composition it operates in the solubilization of a certain level of cannabinoid containing extract in the oral cavity after a subject consumes the non-aqueous composition. The term “ratio” refers to a mass ratio and the term “oil” refers to the mass of the carrier oil for the cannabinoid extract.

As used herein, the term “targeted plurality of emulsifiers to total oil ratio” refers to a measure of the level of emulsifiers to all of the oil present in the emulsification system that a formulator wishes to maintain for the emulsification system so that when it is formulated into a non-aqueous composition it operates in the solubilization of a certain level of cannabinoid containing extract in the oral cavity after a subject consumes the non-aqueous composition. The term “ratio” refers to a mass ratio and the term “total oil” refers to all of the oil present in the emulsification system, such as for example, carrier oil and consumable oils, if present.

As used herein, the term “targeted oil to water ratio” refers to a measure of the level of oil to water in the emulsification system that a formulator wishes to maintain for the emulsification system so that when it is formulated into a non-aqueous composition it operates in the solubilization of a certain level of cannabinoid containing extract in the oral cavity after a subject consumes the non-aqueous composition. The term “ratio” refers to a mass ratio and the term “oil” refers to the mass of the carrier oil for the cannabinoid extract.

As used herein, the term “targeted total oil to water ratio” refers to a measure of the level of all of the oil to water in the emulsification system that a formulator wishes to maintain for the emulsification system so that when it is formulated into a non-aqueous composition it operates in the solubilization of a certain level of cannabinoid containing extract in the oral cavity after a subject consumes the non-aqueous composition. The term “ratio” refers to a mass ratio and the term “total oil” refers to all of the oil present in the emulsification system, such as for example, carrier oil and consumable oils, if present.

As used herein, the terms “preferred”, “preferably” and variants refer to embodiments of the disclosure that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

It is understood that the test methods that are disclosed in the Test Methods Section of the present application must be used to determine the respective values of the parameters of Applicant's disclosures as described and claimed herein.

In all embodiments of the present disclosure, all percentages, parts and ratios are based upon the total weight of the compositions of the present disclosure, unless otherwise specified. All such weights as they pertain to listed ingredients are based on the active level and, therefore do not include solvents or by-products that may be included in commercially available materials, unless otherwise specified.

All ratios are weight ratios unless specifically stated otherwise. All temperatures are in Celsius degrees (° C.), unless specifically stated otherwise. All dimensions and values disclosed herein (e.g., quantities, percentages, portions, and proportions) are not to be understood as being strictly limited to the exact numerical values recited. Instead, unless otherwise specified, each such dimension or value is intended to mean both the recited value and a functionally equivalent range surrounding that value. For example, a dimension disclosed as “40 mm” is intended to mean “about 40 mm.”

A detailed description of one or more of one or more embodiments of the invention is provided below along with an accompanying FIGURE that illustrates the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured

### Emulsification System

In one broad aspect, the present disclosure relates to a cannabinoid based emulsification system having a good cannabinoid solubilization profile. The cannabinoid based emulsification system may also demonstrate enhanced stability, clarity, organoleptic properties and/or slow and/or delayed release of the cannabinoids obtained with use of such emulsification system in non-aqueous compositions to form cannabis infused human or pet edibles and/or confectionaries.

Advantageously, the herein described cannabinoid based emulsification system is compatible with a large variety of different product bases, thereby reducing the need to manufacture multiple cannabinoid based emulsification systems for infusing with a variety of product bases. The herein described cannabinoid based emulsification system may also provide a more consistent user experience when formulated into a large variety of product forms.

The inventors surprisingly discovered that some or all of the above-mentioned benefits can be achieved by selecting a cannabinoid based emulsification system capable of solubilizing the cannabinoids throughout the entire industrial scale production process. This is achieved by formulating the cannabinoids with an emulsification system comprising a plurality of emulsifiers having a targeted combined HLB value, a cannabinoid in a carrier oil having a targeted plurality of emulsifiers to oil ratio, and, optionally, water having a targeted oil to water ratio, such that these components, if present, operate to solubilize at least 10 wt %, preferably at least 20 wt %, preferably at least 30 wt %, preferably at least 50 wt %, or preferably at least 100 wt % of the cannabinoid in the oral cavity within 30 minutes, preferably within 20 minutes, or preferably within 10 minutes after a subject consumes the non-aqueous composition, based on the total weight of the cannabinoid in the non-aqueous composition. Alternatively, the emulsification system according to the present disclosure, wherein it operates to solubilize at least 1 mg, preferably at least 2 mg, preferably at least 5 mg, or preferably at least 10 mg of the cannabinoid in the oral cavity within 30 minutes, preferably within 20 minutes, or preferably within 10 minutes after a subject consumes the non-aqueous composition.

The resultant solubilized cannabinoid emulsions are also stable, preferably over the normal shelf-life of the emulsification system and/or non-aqueous composition. These results are unexpected since previous emulsification approaches incorporating cannabinoids have not focused on such a combination of features to drive cannabinoid solubility in a non-aqueous composition and therefore have not been able to provide some or all of these advantages.

Accordingly, in one aspect the present disclosure is directed to a cannabinoid based emulsification system for infusing a non-aqueous composition with a cannabinoid, the emulsification system comprising: a) at least one cannabinoid in a carrier oil; b) a plurality of self-emulsifiers have a targeted combined HLB value; and c) a targeted plurality of self-emulsifiers to oil ratio; wherein b) and c) operate so that at least 10 wt %, preferably at least 20 wt %, preferably at least 30 wt %, preferably at least 50 wt %, or preferably at least 100 wt % of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

In some embodiments, the emulsification system wherein at most 100 wt %, preferably at most 50 wt %, preferably at most 40 wt %, or preferably at most 30 wt % of the cannabinoid, based on the total weight of the cannabinoid in the non-aqueous composition, is released from the non-aqueous composition within 30 minutes, preferably within 20 minutes, or preferably within 10 minutes after consumption.

In some embodiments, the emulsification system at least 1 mg, preferably at least 2 mg, preferably at least 5 mg, or preferably at least 10 mg of the cannabinoid is absorbed through an oral mucosa surface of the subject within 30 minutes, preferably within 20 minutes, or preferably within 10 minute after consumption.

In some embodiments, the emulsification system according to the present disclosure further comprising: d) water having a targeted oil to water ratio; wherein b), c) and d) operate so that at least 10 wt %, preferably at least 20 wt %, preferably at least 30 wt %, preferably at least 50 wt %, or preferably at least 100 wt % of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

Accordingly, in another aspect the present disclosure is directed to a cannabinoid based emulsification system for infusing a non-aqueous composition with a cannabinoid, the emulsification system comprises: a) at least one cannabinoid in a carrier oil; b) a plurality of emulsifiers having a targeted combined Hydrophile-Lipophile Balance (HLB) value; and c) a targeted plurality of emulsifiers to oil ratio; wherein b) and c) operate so that at least 1 mg, preferably at least 2 mg, preferably at least 5 mg, or preferably at least 10 mg of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

In some embodiments, the emulsification system wherein at most 1 mg, preferably at most 2 mg, preferably at most 5 mg, or preferably at most 10 mg of the cannabinoid, based on the total weight of the cannabinoid in the non-aqueous composition, is released from the non-aqueous composition within 30 minutes, preferably within 20 minutes, or preferably within 10 minutes after consumption.

In some embodiments, the emulsification system according to the present disclosure further comprising: d) water having a targeted oil to water ratio; wherein b), c) and d) so that at operate so that at least 1 mg, preferably at least 2 mg, preferably at least 5 mg, or preferably at least 10 mg of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition. Although not wishing to be bound by theory, it is believed that the cannabinoid based emulsification system of the present disclosure provides the right hydrophilic hydrophobic balance to stabilize the emulsions.

Preferably the emulsification system of the present disclosure is prepared in liquid form, and then spray dried into a powder form. Powder forms may be easier to transport and formulate into a non-aqueous composition, i.e., a solid or dried product form. Spray drying is a conventional chemical process used to produce dry particulate solids from a variety of liquid starting material. Spray drying processes for producing powders are well-known and disclosed, for example, in U.S. Pat. Nos. 5,976,574, 5,985,248, 6,001,336, 6,051,256, 6,077,543, and 6,423,344 and PCT Publications WO 96/32149, WO 99/16419, WO 01/00312, WO 01/85136 and in WO 02/09669, which are incorporated in their entirety by reference. Dry powders obtained using such processes may also be re-hydrated. While spray draying is one method to produce the dry powder, other possible methods may include, for example, a two-step process of (i) pan drying the liquid form, and (ii) then grinding the pan dried material into a fine powder.

In some embodiments, the self-emulsification of the present disclosure is in a powder form, preferably a spray-dried dispersion. According to this embodiment, the self-emulsification system of the present disclosure, wherein upon reconstitution in water forms a stable oil-in-water nano-emulsion. Preferably the resultant nano-emulsion comprises dispersed oil droplets after self-emulsification having a particle size distribution (PSD) of about 200 nm or less, preferably about 100 nm or less, preferably about 80 nm or less, preferably about 60 nm or less, preferably about 40 nm or less, preferably about 20 nm or less, or preferably about 10 nm or less.

Alternatively, the emulsification system of the present disclosure is present in a liquid form for formulating into non-aqueous compositions intended for oral consumption.

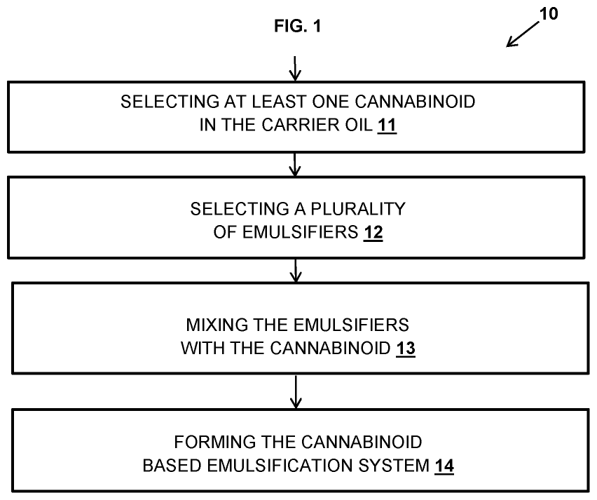

In another aspect, the present disclosure is also directed to a process for manufacturing a cannabinoid based emulsification system as described herein. FIG. 1 shows a flow diagram illustrating an embodiment of a process (10) for preparing a cannabinoid based emulsification system of the present disclosure. At step (11), at least one cannabinoid in the carrier oil is selected based on its suitability for the desired psychoactive or therapeutic effects. At step (12) suitable plurality of emulsifiers are selected. It is desirable to select at least one high HLB value emulsifier and at least one low HLB value emulsifier so as to generate a targeted combined HLB value of the self-emulsifiers according to the present disclosure. The cannabinoid and the emulsifiers selected in steps (11) and (12) are then subjected to a mixing step (13) to enable the formation of the emulsification system in step (14).

Emulsifiers

The HLB value of an emulsifier is related to its solubility. It is well known that an emulsifier having a low HLB value (below 9) will tend to be oil-soluble and one having a high HLB value (above 13) will tend to be water-soluble (HLB: ICI Americas Inc., “The HLB System a Time Saving Guide to Emulsifier Selection”, Chemmunique, March 1980). When two or more emulsifiers are blended, the resulting combined HLB of the emulsifier blend is calculated by summation of the proportion of the HLB from each emulsifier. For instance, the level of an emulsifier multiplied by its unique HLB value provides the contributory HLB value of that specific emulsifier to the combined HLB value of the emulsification system. Previous emulsification approaches involving cannabis have focused on blending emulsifying agents having a combined HLB value in the preferred range of 8-18 without any specificity as to the type of emulsifiers (see U.S. Pat. No. 7,025,992; US 2018/0318399). Other previous emulsification approaches with cannabis have specified blends of at least one high HLB value (at least about 8) surfactant and at least one low HLB value (less than about 5) surfactant (see CA 3,003,120).

Unlike previous proposed uses of emulsification system, the inventors have established that the aforementioned advantages are not tied to the conventional understanding of relying solely on emulsifiers having a particular individual or combined HLB value. Instead, the inventors have discovered new rules to formulate emulsification systems to improve cannabinoid solubility. In particular, the inventors identified the combination of: (i) a targeted combined HLB value of the emulsifiers with at least (ii) a targeted plurality of emulsifiers to oil ratio, and optionally, (iii) a targeted oil to water ratio, so that the emulsification system operates so that at least 10 wt %, preferably at least 20 wt %, preferably at least 30 wt %, preferably at least 50 wt %, or preferably at least 100 wt % or at least 10 wt %, preferably at least 20 wt %, preferably at least 30 wt %, preferably at least 50 wt %, or preferably at least 100 wt % of the cannabinoid is solubilized in the oral cavity within 30 minutes, preferably within 20 minutes, or preferably within 10 minutes after a subject consumes the non-aqueous composition. Also, unlike previous emulsification approaches, the inventors have established that the aforementioned advantages are not tied to a particular HLB type, level or chemistry of the emulsifiers in the emulsification system but can be applied broadly. In fact, what the inventors have established is a systematic approach for formulating cannabinoid based emulsification systems having a longer lasting good cannabinoid solubilization and/or stability profile.

Preferably, the plurality of emulsifiers are present in an amount of from about 0.1 wt % to about 15 wt %, preferably from about 2 wt % to about 12 wt %, based on the total weight of the emulsification system.

Preferably, the plurality of emulsifiers for use in the emulsification systems of the present disclosure may have a targeted combined HLB value that is equal to or greater than 11, preferably in the range of from 11 to 19, preferably in the range of from 11 to 17, or preferably in the range of from 11 to 15. This targeted combined HLB value contributes to better solubilization of the cannabinoids in the oral cavity during consumption. It is also believed to contribute to the stability of the cannabinoid emulsions, particularly as the cannabinoid based self-emulsification system is formulated into a concentrated pre-mix formulation and the eventual non-aqueous product form.

It will be appreciated that the plurality of emulsifiers may comprise of a variety of different types of emulsifiers. In an embodiment, the plurality of emulsifiers for use in the emulsification system may comprise: (i) a high HLB emulsifier, preferably a high HLB non-ionic emulsifier, having an individual HLB value of equal to or greater than 9, preferably in the range of from 9 to 17; and (ii) a low HLB emulsifier, preferably a low HLB non-ionic emulsifier, having an individual HLB value of below 9, preferably in the range of from 1 to 8.

Preferably, the high HLB non-ionic emulsifier is one or more selected from the group consisting of: polysorbates, polyoxyethylenes, polyoxypropylene block co-polymers, ethoxylated aliphatic alkyl alcohols, ethoxylated fatty alcohols, ethoxylated aliphatic alkyl acids, ethoxylated fatty acids, and a combination thereof. Suitable non-limiting examples of high HLB non-ionic-emulsifier include one or more selected from the group consisting of: polyoxyethylene monostearate (PEG 400 Monostearate), polyoxyethylene monooleate (PEG 400 Monoleate), polyoxyethylene sorbitan monolaurate (Tween® 20), polyoxyethylene sorbitan monolaurate (Tween® 21), polyoxyethylene sorbitan monopalmitate (Tween® 40), polyoxyethylene sorbitan monostearate (Tween® 60), polyoxyethylene sorbitan monostearate (Tween® 61), polyoxyethylene sorbitan tristearate (Tween® 65), polyoxyethylene sorbitan monooleate (Tween® 80), polyoxyethylene sorbitan monooleate (Tween 81), polyoxyethylene sorbitan trioleate (Tween® 85), polyoxyethylene-(15)-stearic acid (Pegosperse 1500MS), polyoxyethylene-(20)-stearyl alcohol (Brij 78), polyoxyethylene-(23)-lauryl alcohol (Brij 35), (Lutensol ON 60), PEG-40 hydrogenated castor oil (Cremophor/Kolliphor RH 40), PEG-35 castor oil (Cremophor EL), Solutol HS-15 and a combination thereof, preferably polyoxyethylene sorbitan monooleate (Tween® 80).

The emulsification system of the present disclosure preferably comprises from about 55 wt % to about 99 wt % based on the total weight of the plurality of emulsifiers of the high HLB non-ionic emulsifier.

Preferably, the low HLB non-ionic emulsifier is one or more selected from the group consisting of glyceryl monostearates, sorbitan fatty acid esters, capril caprylic macrogolglycerides, propylene glycol laurates, propylene glycol caprylates, glycerol monostearate, polyglycerol oleates, lecithin-based emulsifiers, tocopherols, polyoxyethylenes, and a combination thereof. Suitable examples of low HLB non-ionic emulsifiers include one or more selected from the group consisting of: sorbitan monopalmitate (Span 40), sorbitan monostearate (Span 60), sorbitan tristearate (Span 65), sorbitan monooleate (Span 80), sorbitan trioleate (Span 85), sunflower lecithin emulsifier, soybean lecithin emulsifier, linseed lecithin emulsifier, olive lecithin emulrapeseed lecithin emulsifier, egg lecithin emulsifier, corn lecithin emulsifier, peanut lecithin emulsifier, algal lecithin emulsifier, Vitamin E and Vitamin E derivatives (alpha, beta, gamma and delta-tocopherols), preferably d-alpha-tocopherol polyethyleneglycol 1000 succinate (Vitamin E TPGS), blend of isomers of alpha-tocopherol, beta-tocopherol, gamma-tocopherol and delta-tocopherol (Tocobiol®), polyoxyethylene (2) cetyl ether (Brij C2), and a combination thereof.

The emulsification system of the present disclosure preferably comprises from about 1 wt % to about 45 wt % based on the total weight of the plurality of emulsifiers of the low HLB non-ionic emulsifier.

Preferably the cannabinoid based emulsification system of the present disclosure comprises a plurality of emulsifiers, wherein a ratio, preferably a mass ratio, of the high HLB non-ionic emulsifier to the low HLB non-ionic emulsifier is in the range of from about 10:1 to about 1:10.

### Cannabinoid

The cannabinoid based emulsification system of the present disclosure comprises at least one cannabinoid. Cannabinoids are commonly used for recreational purposes to produce physiological effects associated with a feeling of physical and/or emotional satisfaction. Cannabinoids can also be useful in the treatment and/or prophylaxis of a wide variety of diseases or conditions, such as pain, anxiety, inflammation, autoimmune diseases, neurological disorder, psychiatric disorder, malignancy, metabolic disorder, nutritional deficiency, infectious disease, gastrointestinal disorder, or cardiovascular disorder. The cannabinoids may also have application as neuroprotectants, for example, in limiting neurological damage following ischemic insults, such as stroke and trauma, or in the treatment of neurodegenerative diseases such as Alzheimer's disease, Parkinson's disease and HIV dementia.

As used herein, the term “cannabinoid” is generally understood to include any chemical compound that acts upon a cannabinoid receptor. For instance, cannabinoids may include endocannabinoids (i.e., produced naturally by humans and animals), phytocannabinoids (i.e., found in cannabis and some other plants), and synthetic cannabinoids (i.e., manufactured artificially).

Suitable examples of phytocannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-ci s-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6 a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (ci s-THC), 3,4,5,6-tetrahydro-7-hydroxy-alpha-alpha-2-trimethyl-9-n-propyl-2, 6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof. Further examples of suitable cannabinoids are discussed in PCT Patent Application Pub. No. WO2017/190249 and U.S. Patent Application Pub. No. US2014/0271940, which are incorporated by reference in their entirety.

Suitable examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

The cannabinoid may be in an acid form or a non-acid form, the latter also being referred to as the decarboxylated form since the non-acid form can be generated by decarboxylating the acid form. Preferably, where reference is made to a particular cannabinoid, it will be understood that the cannabinoid is in the decarboxylated form (i.e., non-acid form).

Suitable examples of source material comprising cannabinoids include, but are not limited to, cannabis or hemp plant material (e.g., flowers, seeds, trichomes, and kief), milled cannabis or hemp plant material, extracts obtained from cannabis or hemp plant material (e.g., resins, waxes and concentrates), and distilled extracts or kief. In some embodiments, pure or isolated cannabinoids and/or source materials comprising cannabinoids may be combined with water, lipids, hydrocarbons (e.g., butane), ethanol, acetone, isopropanol, or mixtures thereof.

In some embodiments, the cannabinoid based emulsification system comprises an effective amount of the cannabinoid for producing physiological effects associated with a feeling of physical and/or emotional satisfaction once formulated into the cannabis infused products (e.g., beverages, human or pet edibles, confectionaries). In some embodiments, the cannabinoid based emulsification system comprises an effective amount of the cannabinoid for treating or alleviating a disease or condition once formulated into the cannabis infused products (e.g., beverages, human or pet edibles, confectionaries). Preferably, the cannabinoid based emulsification system comprises the cannabinoid present in an amount of from about 1 mg/mL to about 50 mg/mL, preferably from about 4 mg/mL to about 40 mg/mL, or preferably from about 10 mg/mL to about 25 mg/mL. Cannabinoid provided at such an amount in the cannabinoid based emulsification system of the present disclosure can be particularly effective in delivering the desired physiological effects and/or treating or alleviating a disease or condition once formulated into the cannabis infused products. Such concentration of cannabinoid in the cannabinoid based emulsification system may also be effective in delivering the desired fast onset of action once formulated into the cannabis infused products.

In some embodiments, the types of cannabinoids and/or the levels of the cannabinoids incorporated into the cannabinoid based emulsification system of the present disclosure provide substantially no psychoactive effect or no psychoactive effect. In other words, the types of cannabinoids and/or the levels of the cannabinoids used in the present cannabinoid based self-emulsification system do not substantially or do not affect mood, perception, consciousness, cognition or behaviour of a subject, as a result of changes in the normal functioning of the nervous system.

In some embodiments, it is desirable that various cannabinoids can be used in combination to achieve the desired effect. Suitable combinations of the cannabinoid which can be used in the present disclosure include a combination of tetrahydrocannabinol (THC), and cannabidiol (CBD). Certain specific ratios of cannabinoids may be useful to produce the feeling of physical and/or emotional satisfaction and/or may be useful in the treatment or management of specific diseases or conditions.

In some aspects, the (w/w) ratio of the THC to the CBD is between about 1:1000 and about 1000:1. Preferably, the (w/w) ratio of THC to CBD in the composition may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1. Preferably, the cannabinoid based self-emulsification system of the present disclosure wherein the cannabinoid is a combination of the cannabidiol (CBD) and the tetrahydrocannabinol (THC) present in a ratio of from about 2:1 to about 1:2.

In another embodiment, the cannabinoid is cannabidiol (CBD), which is a non-psychoactive form of the cannabinoid. The terms “cannabidiol” and “CBD” are used interchangeably and generally understood to refer to one or more of the following compounds, and, unless a particular other stereoisomer or stereoisomers are specified, includes the compound “Δ2-cannabidiol”. These compounds may include for example: (1) Δ5-cannabidiol (2-(6-isopropenyl-3-methyl-5-cyclohexen-1-yl)-5-pentyl-1,3-benzenediol); (2) Δ4-cannabidiol (2-(6-isopropenyl-3-methyl-4-cyclohexen-1-yl)-5-pentyl-1,3-benzenediol); (3) Δ3-cannabidiol (2-(6-isopropenyl-3-methyl-3-cyclohexen-1-yl)-5-pentyl-1,3-benzenediol); (4) Δ3,7-cannabidiol (2-(6-isopropenyl-3-methylenecyclohex-1-yl)-5-pentyl-1,3-benzenediol); (5) Δ2-cannabidiol (2-(6-isopropenyl-3-methyl-2-cyclohexen-1-yl)-5-pentyl-1,3-benzenediol); (6) Δ1-cannabidiol (2-(6-isopropenyl-3-methyl-1-cyclohexen-1-yl)-5-pentyl-1,3-benzenediol); and (7) Δ6-cannabidiol (2-(6-isopropenyl-3-methyl-6-cyclohexen-1-yl)-5-pentyl-1,3-benzenediol).

In another embodiment, the cannabinoid is THC.

In another embodiment, the cannabinoid is an isolated cannabinoid having >90%, preferably >95%, preferably >98%, preferably >98%, preferably >99% or preferably >99.5%, purity present in at least one carrier oil or solvent. It is especially preferred that the cannabinoids have high purity (i.e., Pharmacopoeia Grade substances, which may be obtained from a natural source or via synthetic means) to enable sufficient solubility in the composition. Solubility is important so that the cannabinoids remain in solution and do not precipitate out over time.

The cannabinoid based emulsification system of the present disclosure may further comprise at least one terpenoid. These terpenoids, which are hydrocarbons, are derivatives of terpenes and responsible for not only giving cannabis its distinct aroma and flavor but can alter the “high” experience itself. The cannabinoids and terpenes may interact co-operatively to create what referred to as an “entourage effect” that magnifies the psychoactive or therapeutic benefits of the cannabis plant's individual components so that the medicinal impact of the whole plant is greater than the sum of its parts. The terpenoid compounds added may include, but are not limited to, one or more of any specific class of naturally occurring or synthetic terpenoids such as hemiterpenoids, monoterpenoids, sesquiterpenoids, diterpenoids, sesterterpenoids, triterpenoids, tetraterpenoids, polyterpenoid, and mixtures or derivatives thereof.

### Carrier Oil

Cannabis formulations intended for use in these types of cannabis infused products are typically formulated with a carrier oil (e.g., triglyceride oil such as “medium chain triglyceride” (MCT)), as opposed to aqueous formulations, due to cannabinoids being highly lipophilic (i.e., fat-loving) and having poor water aqueous solubility (e.g., essentially water insoluble). The purpose of the carrier oil is to aid in solubilizing the hydrophobic cannabinoid in the formulation.

The cannabinoid based emulsification system of the present disclosure comprises the cannabinoid in a carrier oil. Non-limiting examples of carrier oils suitable for cannabinoids include: borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (e.g., caprylic/capric glycerides), ethanol, acetone, isopropanol, hydrocarbons or a combination thereof.

The inventors have surprisingly discovered that in addition to the targeted combined HLB value of the emulsifiers, a targeted plurality of emulsifiers to oil ratio is another key factor for cannabinoid solubilization in the emulsification system of the present disclosure. In other words, the inventive emulsification approach formulates an emulsification system having a targeted ratio of the emulsifiers to the carrier oil to enable sufficient solubilization of the cannabinoid in the oral cavity upon consumption of the non-aqueous composition comprising the emulsification system.

In some embodiments, the cannabinoid based emulsification system of the present disclosure, wherein c) the targeted plurality of emulsifiers to oil ratio is from about 4.5:1 to about 1:1, preferably from about 4.3:1 to about 1.5:1. Alternatively, the targeted plurality of emulsifiers to oil ratio may vary outside of this range so long as the formulator can adjust the targeted combined HLB value of the emulsifiers, and optionally along with the targeted oil to water ratio (as discussed further below), so that the emulsification system continues to operate to solubilize the cannabinoid in the oral cavity upon consumption of the non-aqueous composition comprising the emulsification system. The targeted ratio of the plurality of emulsifiers to oil is not tied to a particular HLB type, level or chemistry of the emulsifiers or a particular type of carrier oil.

### Water

The present cannabinoid based emulsification system may include a liquid carrier, such as for example, water, preferably USP water, due to its many benefits. The water may be added as an ingredient on its own right or it may be present as a carrier in other common raw materials. The water content as used herein means the total amount of water present in the cannabinoid based emulsification system, whether added separately or as a solvent or carrier for other raw materials.

The water may be present in an amount of from about 70 wt % to about 95 wt %, preferably from about 75 wt % to about 85 wt %, based on the total weight of the emulsification system.

As previously mentioned, in addition to the targeted combined HLB value of the self-emulsifiers, the inventors have also identified the feature of a targeted oil to water ratio as another key factor for cannabinoid solubilization in the emulsification system of the present disclosure. In other words, the inventive emulsification approach formulates an emulsification system having a targeted ratio of the carrier oil to water to enable sufficient solubilization of the cannabinoid in the oral cavity upon consumption of the non-aqueous composition comprising the emulsification system.

In some embodiments, the cannabinoid based emulsification system of the present disclosure, wherein d) the targeted oil to water ratio is from about 1:30 to about 1:40, preferably from about 1:33 to about 1:37. Alternatively, the targeted oil to water ratio may vary outside of this range so long as the formulator can adjust the targeted combined HLB value of the self-emulsifiers, and the targeted plurality of emulsifiers to oil ratio (as discussed above), so that the emulsification system continues to operate to solubilize at least 1 mg of the cannabinoid in 1 mL of the non-aqueous composition. The targeted ratio of the oil to water is not tied to a particular type of carrier oil or a particular type of water.

### Consumable Oil

The cannabinoid based emulsification system preferably further comprises a consumable oil. The consumable oil may function to further improve the cannabinoid solubilization in the emulsification system because cannabinoids exhibit a high degree of solubility in the consumable oil. The consumable oil may also aid to increase bioavailability of the cannabinoid as it reaches the blood stream more quickly than the carrier oil alone. The use of consumable oil to enhance cannabinoid solubility for edibles and beverages is preferred as they do not adversely influence flavor.

As used herein, the term “consumable oil” means an oil suitable for human or animal consumption. Consumable oils can be hydrogenated oils, chemically or enzymatically interesterified oils, fractionated oils, and blended oils. Suitable non-limiting examples may include: a medium chain triglyceride (e.g., Labrafac™ CC MCT), a coconut oil, a citrus oil (e.g., lemon oil, orange oil), a corn oil, a cottonseed oil, a flax seed oil, a grape seed oil, a marine oil (e.g., a fish oil, an algal oil, a fungal oil), a mustard oil, a nut oil (e.g., almond oil, cashew oil, walnut oil), an olive oil, a palm oil (and fractions), a peanut oil, a rapeseed oil (e.g., a canola oil), a rice bran oil, a safflower oil, a sesame oil, a soybean oil, a sunflower oil, or mixtures thereof. The consumable oil can be used either singly or in combination with one another.

The consumable oil may be present in an amount of from about 0.01% to about 10%, preferably from about 0.1% to about 5%, or preferably from about 0.5% to about 4% by weight of the emulsification system. In another aspect, the consumable oil and the carrier oil may be present in a weight ratio of from 2:1 to 1:2, preferably from 1.5:1 to 1:1.5. Other ratios of the consumable oil to carrier oil are permissible so long as they do not adversely affect the solubilization of the cannabinoids in the oral cavity upon consumption of the non-aqueous composition comprising the emulsification system.

It has also been discovered that a specific targeted plurality of emulsifiers to total oil ratio is important particularly to cannabinoid based emulsification systems of the present disclosure, which are formulated to ensure sufficient solubilization of the cannabinoid in the non-aqueous composition. As used herein, the term “total oil” means the total amount of oil present in the cannabinoid based emulsification system, whether added separately or as a carrier for other materials (e.g., cannabinoids). For example, the total oil may include the carrier oil for the cannabinoids and the additional consumable oils intentionally added to the formulation.

In some embodiments, the cannabinoid based emulsification system of the present disclosure further comprising e) a consumable oil having a targeted plurality of emulsifiers to total oil ratio; wherein b), c) and e) operate so that at least 10 wt %, preferably at least 20 wt %, or preferably at least 30 wt %, or preferably at least 50 wt % of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

In some embodiments, the cannabinoid based emulsification system of the present disclosure further comprising e) a consumable oil having a targeted plurality of emulsifiers to total oil ratio; wherein b), c) and e) operate so that at least 1 mg, preferably at least 2 mg, or preferably at least 5 mg of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

In some embodiments, the cannabinoid based emulsification system of the present disclosure, wherein e) the targeted plurality of emulsifiers to total oil ratio is from about 1:1 to about 2.5:1, preferably from about 1.5:1 to about 2.1:1. Alternatively, the targeted plurality of emulsifiers to total oil ratio may vary outside of this range so long as the formulator can adjust the targeted combined HLB value of the emulsifiers, so that the emulsification system continues to operate to solubilize the cannabinoids in the oral cavity upon consumption of the non-aqueous composition comprising the emulsification system. The targeted plurality of emulsifiers to total oil ratio is not tied to a particular HLB type, level or chemistry of the emulsifiers or a particular type of carrier oil or consumable oil (if present).

It has also been discovered that a specific targeted total oil to water ratio is important particularly to cannabinoid based emulsification systems of the present disclosure, which are formulated to ensure sufficient solubilization of the cannabinoid in the non-aqueous composition. In an embodiment, the cannabinoid based emulsification system of the present disclosure, wherein d) the water having a targeted total oil to water ratio; wherein b), c), d) and e) operate to solubilize the cannabinoids in the oral cavity upon consumption of the non-aqueous composition comprising the emulsification system. Preferably, the targeted total oil to water ratio is from about 1:20 to about 1:10, preferably from about 1:18 to about 1:15. Alternatively, the targeted total oil to water ratio may vary outside of this range so long as the formulator can adjust the targeted combined HLB value of the self-emulsifiers, and the targeted plurality of self-emulsifiers to total oil ratio (as discussed above), so that the self-emulsification system continues to operate to solubilize the cannabinoids in the oral cavity upon consumption of the non-aqueous composition comprising the emulsification system. The targeted total oil to water ratio is not tied to a particular type of carrier oil or consumable oil (if present).

### Other Ingredients

The present cannabinoid based emulsification system can comprise additional ancillary components that are known to one skilled in the art. It will be appreciated that selected components for the cannabinoid based emulsification system are the usual and conventional components known to those skilled in the art, and must be chemically and physically compatible with one another. Suitable non-limiting examples of such ancillary components include antioxidants, preservatives, and stabilizers.

### Cannabinoid Concentrate Composition

The inventors have discovered a simple and reliable mean to dose cannabinoids into a non-aqueous composition by using the cannabinoid based emulsification system as described herein. The solution is to prepare a cannabinoid concentrate composition (i.e., a pre-mix) using the cannabinoid based emulsification system.

The cannabinoid concentrate composition thus prepared is easy to store, transport, and add to any manufacturing line using simple equipment that already exists on food, and confectionaries manufacturing lines, without the need for incurring additional complex material handling capital. It is also easy to distribute the cannabinoid concentrate composition throughout the whole batch homogenously during the manufacturing process by conventional mixing operations. For example, the cannabinoid concentrate composition can be dissolved into solid edibles or confectionaries.

Preferably, the cannabinoids in the emulsification system of the present disclosure are microencapsulated in an emulsion. Microencapsulation techniques may include the emulsification techniques involving mixing, homogenization, injection, spray drying, spray cooling, spray chilling, freeze-drying, air suspension coating, fluidized-bed extrusion, centrifugal extrusion, coacervation, rotational suspension separation, cocrystallization, liposome entrapment, interfacial polymerization, molecular inclusion, microfluidization, ultrasonication, physical adsorption, complex formation, nanosized self-assembly, or any combination thereof. The microencapsulation process may be assisted or accelerated by the application of heat (e.g., through microwave irradiation). Mixing may be modelled using idealized chemical reactors, which may include, but are not limited to, batch reactors, continuous stirred-tank reactors, and plug flow reactors.

The cannabinoids are formulated as suspended droplets within the emulsion surrounded by one or more bilayers, preferably lipid bilayers (e.g., droplets contained in a core). Preferably, the emulsion is formulated as an oil-in-water emulsion having a dispersed phase comprising oil droplets, which can have micro- or nano-particle size distributions. Particle size distribution in an emulsion is an important parameter which contributes to solubilization of cannabinoids, control of delayed release of the cannabinoids, specific turbidity and/or creaming stability of an emulsion. It is understood that these properties can be improved by controlling the particle size distributions.

It is also important to note that it is particularly challenging to maintain particle size distributions from the oil-in-water emulsions containing the cannabinoids. Without wishing to be bound by theory this is primarily due to the effect called Ostwald Ripening. Ostwald Ripening is the phenomena often found in oil-in-water emulsions in which smaller oil particles in solution spontaneously dissolve and deposit on larger oil particles to reach a more thermodynamically stable state wherein the surface area to volume ratio is minimized. The combination of destabilization by oil droplet collisions and coalescence, in addition to Ostwald Ripening in the case of volatile oils, can lead to the oil phase eventually becoming one big droplet to lower surface energy and minimize total surface area. When this occurs, over time the emulsion becomes unstable and eventually two separate phases. The inventors have solved this problem by formulating with the cannabinoid based emulsification system as described herein.

Accordingly, the present disclosure also relates to a cannabinoid concentrate composition in the form of an emulsion, the composition comprising a cannabinoid based emulsification system as described herein and the emulsion having a particle size distribution (PSD) of less than 1000 nm, preferably 200 nm or less, preferably 100 nm or less, preferably 80 nm or less, preferably 60 nm or less, preferably 40 nm or less, preferably 20 nm or less. The term “particle size”, as used herein, refers to a volume based particle size measured, for example, by laser diffraction method. Laser diffraction measures particle size distribution by measuring the angular variation in intensity of light scattered as a laser beam passes through a dispersed particulate sample. Large particles scatter light at small angles relative to the laser beam and small particles scatter light at large angles. The angular scattering intensity data is then analyzed to calculate the size of the particles responsible for creating the scattering pattern, using the Mie theory of light scattering. The particle size is reported as a volume equivalent sphere diameter. Alternatively, the PSD can be measured by laser diffraction according to ISO 13320:2009 and ISO 9276-2:2014.

In some embodiments, the cannabinoid concentrate composition of the present disclosure, wherein the cannabinoids may be microencapsulated in an oil-in-water nano-emulsion, preferably comprising oil droplets having a PSD of about 100 nm or less, preferably about 80 nm or less, preferably about 60 nm or less, preferably about 50 nm or less, or preferably from about 30 nm to about 80 nm.

In some embodiments, the cannabinoid concentrate composition of the present disclosure, wherein the cannabinoids may be microencapsulated in an oil-in-water nano-emulsion comprising oil droplets having a D₉₀ of about 100 nm or less, preferably about 80 nm or less, preferably about 60 nm or less, preferably about 50 nm or less, or preferably from about 30 nm to about 80 nm. The term “D₉₀” means the particle size of no more than 90% of the total amount of particles. For example, a D₉₀ of 100 nm or less means that no more than 90% of the total amount of particles may have a particle size of 100 nm or less.

In some embodiments, the cannabinoids may be microencapsulated in the oil-in-water nano-emulsion comprising oil droplets having a D₅₀ of about 100 nm or less, preferably about 80 nm or less, preferably about 60 nm or less, or preferably about 50 nm or less, or preferably from about 30 nm to about 80 nm. The term “D₅₀” means the particle size of no more than 50% of the total amount of particles. For example, a D₅₀ of 100 nm or less means that no more than 50% of the total amount of particles may have a particle size of 100 nm or less.

It is desirable that the produced cannabinoid concentrate composition has at least 1 month, preferably at least 4 months, preferably at least 6 months, preferably at least 12 months or preferably at least 24 months shelf-life or phase stability (i.e., no visible phase separation). By stability herein is meant that the emulsion formed from the cannabinoid in the carrier oil or solvent is stable against phase separation under storage conditions up to 40-50° C.

In some embodiments, the cannabinoid concentrate composition of the present disclosure, wherein the cannabinoids may be microencapsulated in the oil-in-water nano-emulsion comprising oil droplets wherein the PSD remains substantially similar or the same after a storage period of at least 1 day, preferably after at least 1 week, preferably after at least 1 month or preferably after at least 2 months at 40° C.

In some embodiments, the cannabinoid concentrate composition of the present disclosure, wherein the cannabinoids may be microencapsulated in the oil-in-water nano-emulsion comprising oil droplets having a PSD of about 100 nm or less, preferably about 80 nm or less, preferably about 60 nm or less, preferably about 50 nm or less, or preferably from about 30 nm to about 80 nm, after a storage period of at least 2 weeks, preferably after at least 1 month, or preferably after at least 2 months at 40° C.

The cannabinoid concentrate composition according to the present disclosure may include one or more other components such as, for example, a co-solvent, a preservative, or a buffering agent.

Preferably, the pH of the cannabinoid concentrate composition of the present disclosure is from about 5 to about 10, preferably from about 6 to about 8, or preferably from about 6.5 to about 7.5. The cannabinoid concentrate composition may include one or more pH-adjusting agents to improve solubility and/or stability. It is believed that the pH modifiers can also aid with cannabinoid release during consumption.

The pH of the cannabinoid concentrate composition may be modified using any pharmaceutically acceptable means. Suitable examples of pH modifiers include, but are not limited to, organic acid or base, preferably tartaric acid, phosphoric acid, hydrochloric acid, maleic acid, sodium hydroxide, citric acid and the like known to those of ordinary skill in the art.

The pH is typically measured using a ratio of 1:3 of composition:water, whereby 1 gram of the composition is mixed into 3 grams of deionized water, and then the pH is assessed with an industry accepted pH probe that is calibrated under ambient conditions. The pH is measured by a pH meter with Automatic Temperature Compensating (ATC) probe. The pH meter is capable of reading to 0.001 pH unit.

After each usage the electrode should be washed free from the sample solution with water. Remove any excess water by wiping with a tissue, such as Kimwipes™ or equivalent. When electrode is not in use, keep electrode tip immersed in pH 7 buffer solution or electrode storage solution. Equipment details are as follows:
pH Meter: Meter capable of reading to 0.01 or 0.001 pH units.
Electrode: Orion Ross Sure-Flow combination: Glass body—VWR #34104-834/Orion #8172BN or VWR #10010-772/Orion #8172BNWP.
   Epoxy body—VWR #34104-830/Orion #8165BN or VWR #10010-770/Orion #8165BNWP.
   Semi-micro, epoxy body—VWR #34104-837/Orion #8175BN or VWR #10010-774/Orion #3175BNWP.
   Orion PerpHect combination: VWR #34104-843/Orion #8203BN semi-micro, glass body.
ATC Probe: Fisher Scientific, Cat. #13-620-16.

In some embodiments, the cannabinoid concentrate composition is in a liquid form. Preferably, a liquid cannabinoid concentrate composition provided herein may be clear or transparent. The appearance of a liquid cannabinoid concentrate composition depends on the scattering of light by the droplets.

Preferably, for clear liquid cannabinoid concentrate composition, the majority of the droplets should be less than about 100 nm, preferably less than about 90 nm, preferably less than about 80 nm, preferably less than about 70 nm, preferably less than about 60 nm, or preferably less than about 50 nm in diameter so that light scattering is weak. The cannabinoid concentrate composition of the present disclosure preferably has a turbidity (i.e., cloudiness) of about 30 Nephelometric Turbidity Units (NTU) or less, preferably about 25 NTU or less, or preferably about 20 NTU or less.

It is desirable that the liquid cannabinoid concentrate composition of the present disclosure remains clear over the normal shelf-life (i.e., at least 6 months). In a specific embodiment, the liquid cannabinoid concentrate composition is transparent or translucent, preferably after a storage period of at least 2 weeks, preferably after at least 1 month, or preferably after at least 2 months at 40° C. In an alternative embodiment, the liquid cannabinoid concentrate composition of the present disclosure does not contain visible particles, does not contain visible crystals, does not exhibit phase separation, and/or does not exhibit ringing, preferably after a storage period of at least 2 weeks, preferably after at least 1 month, or preferably after at least 2 months at 40° C.

In some embodiments, the cannabinoid concentrate composition is prepared in a liquid form, and then spray dried into a powder form. The spray drying is a conventional chemical process and similar to the process used to produce the powder form of the cannabinoid based emulsification system as described herein above.

The cannabinoid concentrate composition according to the present disclosure can be made via a number of different processes. In an embodiment, the present disclosure is directed to a process to make the cannabinoid concentrate composition as described herein, comprising the steps of:
a) providing an oil phase comprising the cannabinoid extract;
b) preparing an aqueous phase comprising water and optionally other ingredients;
c) incorporating the emulsification system as described herein either in the oil phase or in the aqueous phase; and
d) dispersing the oil phase in the aqueous phase to form an emulsion, preferably a nano-emulsion.

Preferably, the process further comprises:
e) forming a nano-emulsion by sonicating, high pressure homogenizing, mixing, or a combination thereof of the emulsion of step d).

### Non-Aqueous Composition

The inventors have surprisingly discovered an improved way for the large scale production of cannabis infused human or pet edibles and/or confectionaries that enhances water solubility of the cannabinoids, improves stability of the cannabinoids, provides superior organoleptic profiles, and/or delays release of the cannabinoids upon consumption by a subject. As noted above, this approach is also easy to implement with existing manufacturing lines over a range of different industries, and the intermediates and final products are easy to store and transport, with minimal or no negative impact on the properties as noted above.

Essentially, the solution is to formulate a non-aqueous composition comprising the cannabinoid concentrate composition, which further comprises the cannabinoid based emulsification system as described herein.

In some embodiments, the non-aqueous composition is formed into an edible product intended for human consumption. An edible product can be any product that is suitable, e.g., non-toxic, for placing into the mouth of a human, whether ingested, absorbed, or only chewed or sucked on and at least a portion discarded, etc. Illustrative examples of human edible products include chewing or bubble gums, mints, suckers, jawbreakers, lozenges, hard candies, gummy candies, taffies, chocolates, brownies, cookies, crackers, granola or meal replacement bars, smokeless inhalation powders, honey, syrup, spreads, and dissolving strips. Preferably, the human edible products include gums, hard candies, soft candies, gummy candies, jellies, or lozenges, more preferably chewing or bubble gum or mints.

In some embodiments, the amount of the cannabinoids in a human edible product is enough to produce noticeable psychoactive effects associated with cannabinoids in a subject consuming at least a recommended amount of the edible product. Generally, a recommended amount is an amount that will produce psychoactive effects but not so great as to cause undesirable side effects or toxic effects.

In some embodiments, the amount of the cannabinoids in a human edible product is enough to produce a therapeutic and/or prophylactic effect associated with cannabinoids in a subject consuming at least a recommended amount of the edible product. For example, a recommended amount is an amount that will produce reduction in the feeling of anxiety.

Preferably, the human edible product does not have a perceptible cannabis odor to a subject consuming the edible.

In some embodiments, the non-aqueous composition is formed into an edible product intended for animal consumption. Preferably, the composition of the present disclosure is formed into an edible pet product, preferably an edible pet food or an edible pet chew. Preferably, the non-aqueous composition is a solid form comprising a coating, and wherein the coating is a delayed release coating, a sustained release coating or an immediate release coating.

In some embodiments, the compositions of the present disclosure have a delayed release of the cannabinoids upon consumption by a subject.

### TEST METHODS

The following assays set forth must be used in order that the invention described and claimed herein may be more fully understood.

### Test Method 1: Dynamic Light Scattering (DLS) Method

The particle sizes of the cannabinoid emulsions of the present disclosure are measured in a water solution at 25° C. using the Dynamic Light Scattering (DLS) method. All samples are analyzed at a dilution of 1/20 in purified water and measurements require 1-2 mL of sample in order to accurately generate a signal. The LiteSizer™ (Anton Paar) particle size analyser was used for all particle size measurements.

### Test Method 2: Stability Study

The purpose of this study is to assess the stability of cannabinoid emulsions of the present disclosure. Cannabinoid emulsions were formulated to a concentration of 20 mg/mL THC (i.e., “concentrate”) and then diluted 100 fold THC (i.e., 0.20 mg/mL, also referred to as “diluted”) in water. Since cannabinoids are known to be prone to oxidation when exposed to atmosphere or immersed in water over time, the inventors were interested in examining whether cannabinoid oxidation could be mitigated through incorporation into emulsions of the present disclosure and whether stability was impacted by the particle size. As a result, additional samples of the cannabinoid emulsions similar to the ones above were prepared with the additional component of antioxidants, typically required to help prevent cannabinoid oxidation.

All samples were prepared in glass containers and incubated at 25° C. (i.e., to mimic long term storage condition) and 40° C. (i.e., to mimic accelerated storage condition) for periods of 2 weeks, 4 weeks, 8 weeks and 12 weeks. The concentration of the cannabinoid emulsion samples are recorded at time zero, 2 weeks, 4 weeks, 8 weeks and 12 weeks. The concentration of the cannabinoid emulsion samples at time zero was compared against subsequent time points to assess stability.

### EXAMPLES

The following examples are provided to further illustrate the present invention and are not to be construed as limitations of the present invention, as many variations of the present invention are possible without departing from its spirit or scope.

### Example 1: Cannabinoid Based Emulsification Systems

Inventive cannabinoid based emulsions having a particle size of 40 nm and 200 nm and having the targeted combined HLB value, targeted plurality of emulsifiers to oil ratio, and targeted oil to water ratio within the scope of the present disclosure are provided below in Tables 1a and 2a. Cannabinoid based emulsions having a particle size of >1000 nm were prepared based on the formulae set out in Tables 1a and 2a, without the additional sonication step. These exemplary formulations span the range from nano-emulsions to macro-emulsions. The foregoing emulsions were prepared as follows:
1. The water and oil phase ingredients were solubilized separately using heat and stirring. In particular, the water phase is comprised of water, Tween® 80, ascorbic acid and EDTA and mixed at 60° C. with a magnetic stir bar for 30 minutes. The oil phase is comprised of Labrafac™ lipophile WL 1349, Tocobiol®, lecithin and THC distillate and mixed at 60° C. with a magnetic stir bar for 30 minutes
2. Once the respective water and oil phases have been prepared they were combined while mixing with a high shear homogenizer at 8000-10000 rpm. The oil phase was added slowly to the water phase over 5 minutes and once completely the resultant emulsion was mixed for an additional 15 minutes. The resultant mixture is a macro-emulsion with a particle size >1000 nm.
3. To generate the 40 nm and 200 nm nano-emulsions, high energy sonication was applied to the macro-emulsions for 10 minutes with 100% amplitude using an LSP-500 Ultrasonic Processor (Sonomechanics, Florida, USA).

The targeted combined HLB value for these cannabinoid nano-emulsions were calculated and summarized in Tables 1a and 2a. The targeted plurality of emulsifiers to oil ratio for these cannabinoid nano-emulsions were calculated and summarized in Tables 1b and 2b. The targeted oil to water ratio for these cannabinoid nano-emulsions were calculated and summarized in Tables 1c and 2c.

TABLE 1a
Cannabinoid based Nano-Emulsion (40 nm) - Targeted Combined
HLB

| | | | | | | Targeted |
|---|---|---|---|---|---|---|
| | | | | | | Combined |
| | Mass | % | % | % | Emuls. | Emuls. |
| Ingredients | (g) | Blend | Oil | Emuls. | HLB | HLB |
| THC | 18.75 | 2.50 | 48.39 | — | — | — |
| Distillate- | | | | | | |
| 03 (g) | | | | | | |
| Labrafac ™ | 20.00 | 2.67 | 51.61 | — | — | — |
| lipophile WL | | | | | | |
| 1349 | | | | | | |
| Ascorbic acid | 4.50 | 0.60 | — | — | — | — |
| Tocobiol ® | 3.75 | 0.50 | — | 4.76 | 8 | 0.38 |
| EDTA | 0.10 | 0.01 | — | — | — | — |
| Lecithin | 15.00 | 2.00 | — | 19.05 | 8 | 1.52 |
| Tween ® 80 | 60.00 | 8.00 | — | 76.19 | 15 | 11.43 |
| Water (g) | 627.90 | 83.72 | — | — | — | — |
| Total | 750.00 | 100.00 | 100.00 | 100.00 | 31 | 13.33 |

TABLE 1b
Cannabinoid based Nano-Emulsion (40 nm) - Targeted
Plurality of Emulsifiers to Oil Ratio

| Total | THC Carrier | Targeted plurality of |
|---|---|---|
| Emulsifiers | Oil | emulsifiers to Oil Ratio |
| 10.5 | 2.50 | 4.2:1 |

TABLE 1c
Cannabinoid based Nano-Emulsion (40 nm) - Targeted
Oil to Water Ratio

| THC Carrier | | |
|---|---|---|
| Oil | Water | Targeted Oil to Water |
| 2.50 | 83.72 | 1:33.5 |

TABLE 2a
Cannabinoid based Nano-Emulsion (200 nm) - Targeted Combined
HLB

| | | | | | | Targeted |
|---|---|---|---|---|---|---|
| | | | | | | Combined |
| | Mass | % | % | % | Emuls. | Emuls. |
| Ingredients | (g) | Blend | Oil | Emuls. | HLB | HLB |
| THC | 18.75 | 2.50 | 48.39 | — | — | — |
| Distillate- | | | | | | |
| 03 (g) | | | | | | |
| Labrafac ™ | 20.00 | 2.67 | 51.61 | — | — | — |
| lipophile WL | | | | | | |
| 1349 | | | | | | |
| Ascorbic acid | 3.75 | 0.50 | — | — | — | — |
| Tocobiol ® | 4.50 | 0.60 | — | 13.04 | 8 | 1.04 |
| EDTA | 0.10 | 0.01 | — | — | — | — |
| Lecithin | 10.00 | 1.33 | — | 34.78 | 8 | 2.78 |
| Tween ® 80 | 15.00 | 2.00 | — | 52.17 | 15 | 7.83 |
| Water (g) | 677.90 | 90.39 | — | — | — | — |
| Total | 750.00 | 100.00 | 100.00 | 100.00 | 31 | 11.65 |

TABLE 2b
Cannabinoid based Nano-Emulsion (200 nm) - Targeted
Plurality of Emulsifiers to Oil Ratio

| Total | THC Carrier | Targeted plurality of |
|---|---|---|
| Emulsifiers | Oil | emulsifiers to Oil Ratio |
| 3.93 | 2.50 | 1.57:1 |

TABLE 2c
Cannabinoid based Nano-Emulsions (200 nm) -
Targeted Oil to Water Ratio

| THC Carrier | | |
|---|---|---|
| Oil | Water | Targeted Oil to Water |
| 2.50 | 90.39 | 1:36 |

The inventors discovered that different particle sizes of the emulsions were achieved by tuning: i) the ratio of the plurality of emulsifiers present in the emulsification system, ii) the ratio of the emulsifiers to the oil, and optionally iii) the ratio of the oil to the water. In particular, they found that a higher concentration of the high HLB value emulsifiers (e.g., Tween® 80) relative to the low HLB value emulsifiers (e.g., Lecithin, Tocobiol®) generated smaller particle size (e.g., 40 nm) nano-emulsions. Conversely, the inventors discovered that a higher concentration of the low HLB value emulsifiers relative to the high HLB value emulsifiers resulted in the larger particle size (e.g., 200 nm) nano-emulsions. The results clearly demonstrate that the emulsification approach of the present disclosure allows for tuning the ratio of the emulsifiers to achieve different particle sizes suitable for formulating with a variety of product bases. Additionally, it eliminates the experimental uncertainty that would normally be associated with using different emulsifier combinations to achieve different particle sizes.

### Example 2: Stability Study of Cannabinoid Based Emulsification Systems

The stability of the cannabinoid based emulsification systems of the present disclosure is assessed using the Stability Study test described herein for cannabinoid emulsions having particle sizes of 40 nm, 200 nm, and >1000 nm as described in Example 1 above. The cannabinoid used in these formulations is THC. Furthermore, antioxidants were also added to certain of these THC emulsions. In parallel, comparative concentrate and diluted compositions having particle size of 40 nm without antioxidants were also prepared. 2-6 samples of the cannabinoid emulsions were tested per condition. The foregoing cannabinoid emulsions are produced through mixing of the components (per Example 1 above). Details of the resultant cannabinoid emulsions are summarized in Table 3. The THC concentration of resultant cannabinoid emulsion samples were measured at initial time 0 (“T0”) and at time 2 weeks (“T2”) after storage at 25° C. The results are then averaged and summarized in Table 3.

TABLE 3
Concentrated and Diluted Emulsion Samples

| | | | | | | THC | | THC | |
|---|---|---|---|---|---|---|---|---|---|
| | Cannabinoid | | Particle | | | Concentration | | Concentration | |
| | Emulsion | | Size | | Antioxidants | (T0) | | (T2) | |
| Sample 1 | 20 | mg/mL | 40 | nm | — | 19.3 | mg/mL | 19.4 | mg/mL |
| | THC | | | | | | | | |
| Sample 2 | 0.20 | mg/mL | 40 | nm | — | 0.115 | mg/mL | 0.112 | mg/mL |
| Sample 3 | 20 | mg/mL | 40 | nm | Yes | 19.5 | mg/mL | 19.9 | mg/mL |
| Sample 4 | 0.20 | mg/mL | 40 | nm | Yes | 0.122 | mg/mL | 0.116 | mg/mL |
| Sample 5 | 20 | mg/mL | 200 | nm | Yes | 19.3 | mg/mL | 19.7 | mg/mL |
| Sample 6 | 0.20 | mg/mL | 200 | nm | Yes | 0.094 | mg/mL | 0.077 | mg/mL |
| Sample 7 | 20 | mg/mL | >1000 | nm | Yes | 15.2 | mg/mL | 23.7 | mg/mL |
| Sample 8 | 0.20 | mg/mL | >1000 | nm | Yes | 0.159 | mg/mL | 0.123 | mg/mL |

The results demonstrate that all concentrate (i.e., 20 mg/mL THC) cannabinoid emulsions maintained stable levels of THC with or without the presence of antioxidants at T0 and T2. From the data it can be concluded that a stable product can be obtained in the absence of antioxidants by incorporating the THC (i.e., cannabinoid) into the inventive emulsion systems of the present disclosure. Furthermore, the data shows that the oil droplet particle size does not affect the stability, as all samples in general showed the same behaviour.

Measurements of the diluted (i.e., 0.20 mg/mL THC) samples at T2 showed a significant drop from the T0 values, where THC concentration ranges from 0.094-0.159 mg/mL. The lower T0 values may be indicative of increased oxidation when diluted with water. Comparison of T0 and T2 for diluted samples show a decrease in THC content, which further hints at cannabinoid instability in a diluted water medium.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

Elements of the composition of the disclosure described in connection with the examples apply mutatis mutandis to other aspects of the disclosure. Therefore, it goes without saying that the compositions of the present disclosure encompass any composition comprising any of the ingredients cited herein, in any embodiment wherein each such ingredient is independently present in any appropriate amount as defined herein. Many such compositions, than what is specifically set out herein, can be encompassed.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any disclosure disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the scope of the present disclosure. It is therefore intended to cover in the appended claims all such changes and modifications that are within the scope of this disclosure.

1.-56. (canceled)

57. A cannabinoid based emulsification system for infusing a non-aqueous composition with a cannabinoid, the emulsification system comprising:
a) at least one cannabinoid in a carrier oil;
b) a plurality of emulsifiers having a targeted combined Hydrophile-Lipophile Balance (HLB) value; and
c) a targeted plurality of emulsifiers to oil ratio;
wherein b) and c) operate so that at least 10 wt %, preferably at least 20 wt %, preferably at least 30 wt %, or preferably at least 50 wt %, of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

58. The emulsification system according to claim 57, wherein at most 50 wt %, preferably at most 40 wt %, or preferably at most 30 wt % of the cannabinoid, based on the total weight of the cannabinoid in the non-aqueous composition, is released from the non-aqueous composition within 30 minutes, preferably within 20 minutes, or preferably within 10 minutes after consumption.

59. The emulsification system according to claim 57, wherein at least 1 mg, preferably at least 2 mg, or preferably at least 5 mg of the cannabinoid is absorbed through an oral mucosa surface of the subject within 30 minutes, preferably within 20 minutes, or preferably within 10 minute after consumption.

60. The emulsification system according to claim 57, further comprising:
d) water having a targeted oil to water ratio;
wherein b), c) and d) operate so that at least 10 wt %, preferably at least 20 wt %, preferably at least 30 wt %, or preferably at least 50 wt % of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

61. A cannabinoid based emulsification system for infusing a non-aqueous composition with a cannabinoid, the emulsification system comprising:
a) at least one cannabinoid in a carrier oil;
b) a plurality of emulsifiers having a targeted combined Hydrophile-Lipophile Balance (HLB) value; and
c) a targeted plurality of emulsifiers to oil ratio;
wherein b) and c) operate so that at least 1 mg, preferably at least 2 mg, or preferably at least 5 mg of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

62. The emulsification system according to claim 61, wherein at most 1 mg, preferably at most 2 mg, or preferably at most 5 mg of the cannabinoid, based on the total weight of the cannabinoid in the non-aqueous composition, is released from the non-aqueous composition within 30 minutes, preferably within 20 minutes, or preferably within 10 minutes after consumption.

63. The emulsification system according to claim 61, further comprising:
d) water having a targeted oil to water ratio;
wherein b), c) and d) so that at operate so that at least 1 mg, preferably at least 2 mg, or preferably at least 5 mg of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

64. The emulsification system according to claim 57, wherein:
b) the targeted combined HLB value is equal to or greater than 11, preferably from 11 to 19, preferably from 11 to 17, or preferably from 11 to 15.

65. The emulsification system according to claim 57, wherein:
b) the plurality of the emulsifiers comprise: (i) a high HLB non-ionic emulsifier having an HLB value of 9 to 17, and (ii) a low HLB non-ionic emulsifier having an HLB value of 1 to 8.

66. The emulsification system according to claim 65, wherein:
b) a ratio of the high HLB non-ionic emulsifier to the low HLB non-ionic emulsifier is from about 10:1 to about 1:10.

67. The emulsification system according to claim 57, wherein:
c) the targeted plurality of emulsifiers to oil ratio is from about 4.5:1 to about 1:1, preferably from about 4.3:1 to about 1.5:1.

68. The emulsification system according to claim 63, wherein:
d) the targeted oil to water ratio is from about 1:30 to about 1:40, preferably from about 1:33 to about 1:37.

69. The emulsification system according to claim 61, further comprising
e) a consumable oil having a targeted plurality of emulsifiers to total oil ratio;
wherein b), c) and e) operate so that at least 10 wt %, preferably at least 20 wt %, or preferably at least 30 wt %, or preferably at least 50 wt % of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

70. The emulsification system according to claim 61, further comprising
e) a consumable oil having a targeted plurality of emulsifiers to total oil ratio;
wherein b), c) and e) operate so that at least 1 mg, preferably at least 2 mg, or preferably at least 5 mg of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

71. The emulsification system according to claim 69, wherein:
e) the targeted plurality of emulsifiers to total oil ratio is from about 0.1:1 to about 2.5:1, preferably from about 0.5:1 to about 2.1:1.

72. The emulsification system according to claim 69, wherein:
d) the water having a targeted total oil to water ratio;
wherein b), c), d) and e) operate so that at least 10 wt %, preferably at least 20 wt %, preferably at least 30 wt %, preferably at least 50 wt %, or preferably at least 100 wt % of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

73. The emulsification system according to claim 70, wherein:
d) the water having a targeted total oil to water ratio;
wherein b), c), d) and e) operate so that at least 1 mg, preferably at least 2 mg, preferably at least 5 mg, or preferably at least 10 mg of the cannabinoid is solubilized in the oral cavity within 30 minutes, preferably within 20 minutes, or preferably within 10 minutes after a subject consumes the non-aqueous composition, based on the total weight of the cannabinoid in the non-aqueous composition.

74. The emulsification system according to claim 72, wherein:
d) the targeted total oil to water ratio is from about 1:20 to about 1:10, preferably from about 1:18 to about 1:15.

75. A cannabinoid concentrate composition in the form of an emulsion, the composition comprising a cannabinoid based emulsification system as defined in claim 57, and the emulsion having a particle size distribution (PSD) of less than 1000 nm.

76. The cannabinoid concentrate composition according to claim 75, wherein the emulsion is an oil-in-water nano-emulsion comprising oil droplets having a PSD of about 100 nm or less, preferably about 80 nm or less, preferably about 60 nm or less, preferably about 50 nm or less, or preferably from about 30 nm to about 80 nm.

77. The cannabinoid concentrate composition according to claim 75, wherein the PSD remains substantially similar or the same after a storage period of at least 1 day, preferably after at least 1 week, preferably after at least 1 month or preferably after at least 2 months at 40° C.

78. The cannabinoid concentrate composition according to claim 75, having a pH value ranging from about 5 to about 10, preferably from about 6 to about 8, or preferably from about 6.5 to about 7.5.

79. The cannabinoid concentrate composition according to claim 76, wherein the composition has a turbidity of about 30 Nephelometric Turbidity Units (NTU) or less, preferably about 25 NTU or less, or preferably about 20 NTU or less.

80. The cannabinoid concentrate composition according to claim 76, wherein the composition is transparent or translucent, preferably after a storage period of at least 2 weeks, preferably after at least 1 month, or preferably after at least 2 months at 40° C.

81. A non-aqueous composition comprising a cannabinoid concentrate composition as defined in claim 75.

82. A process to make a cannabinoid concentrate composition comprising the steps of:
a) providing an oil phase;
b) preparing an aqueous phase comprising water and optionally other ingredients;
c) incorporating the emulsification system as defined in claim 57 either in the oil phase or in the aqueous phase; and
d) dispersing the oil phase in the aqueous phase to form an emulsion.

83. The process according to claim 82, further comprising:
e) forming a nano-emulsion by sonicating, high pressure homogenizing, mixing or a combination thereof of the emulsion of step d).
